(19) United States
(12) Patent Application Publication
(10) Pub. No.: US 2009/0043269 A1
(43) Pub. Date: Feb. 12, 2009

(54) APPARATUS FOR DISPENSION OF LIQUID

(76) Inventors: Mikkel J.R. Skou, Copenhagen (DK); Anders Lassen, Frederiksberg (DK)

Correspondence Address: KLEIN, O'NEILL & SINGH, LLP, 43 CORPORATE PARK, SUITE 204, IRVINE, CA, 92606, US

(21) Appl. No.: 11/814,156

(22) PCT Filed: Jan. 20, 2006
(86) PCT No.: PCT/DK2006/000033

§ 371 Date: Sep. 24, 2008

(30) Foreign Application Priority Data
Jan. 20, 2005 (DK) PA 2005 00103

(51) Int. Cl. A61H 35/02; A61F 9/00

(52) U.S. Cl. 604/298; 222/95; 222/566; 604/300

(57) ABSTRACT

A liquid dispensing apparatus includes a liquid storage space having an opening for dispensing liquid, and a movable wall operable to reduce the liquid storage space. The apparatus also includes an intermediate storage space having variable volume, the intermediate storage space including a liquid inlet in fluid communication with the liquid storage space for receiving liquid therefrom. A specific amount of the liquid is transferred from the liquid storage space to the intermediate storage space in a single operation. A nozzle is in fluid communication with the intermediate storage space, the nozzle having an outlet for dispensing the liquid from the intermediate storage space to an intended surface.

The present invention relates to apparatuses for dispensing a liquid from a container into or onto the human eye. The liquid may be of an appropriate composition that may remain sterile for a long period of time, and may be constituted by water including a solution of NaCl, i.e. saltwater. The liquid may alternatively include a medical compound for treating diseases of the eye, such as post-operation treatment compounds and/or disinfecting compounds or and/or antibiotics.

The device according to the present invention may be used for applying liquids to the eye of a patient, e.g. in a period of time after eye surgery. The patient may have hesitations for using apparatuses such as a soft flexible bottle with a liquid dispensing tip where the patient outs his or her head back and holds the bottle in one hand while squeezing the bottle to dispense a drop of liquid into an eye that have recently been operated on.

Further more, the present invention provides an apparatus for dispensing liquid in one easy operation, thereby allowing individuals having disabilities and/or diseases, such as scleroses, arthritis and/or rheumatism to apply liquid to his or her eye without having to perform any twists or turns of the device.

The apparatus or device according to the present invention may also be used to apply liquid, such as saltwater, to an eye or eyes of people in aeroplanes. The air inside an aeroplane, e.g. during long flights, tends to dry out passengers eyes and thereby cause irritation.

Further the liquid may be water including a substance for enhancing the glare of the eye.

Related techniques, apparatuses and devices have been described in previous patent publications such as WO 02/32371 A1, WO 01/45632 A1, WO 93/20785, US 2004/0078010 A1, U.S. Pat. Nos. 4,740,206, 3,756,478, US 2002/0070230 A1, EP 1,179,332, EP 0 437953, U.S. Pat. Nos. 4,623,337, 5,207,569 and US 2003/078551. All of the above mentioned patent publications are hereby incorporated in the present description by reference in their entirety and for all purposes.

A first aspect of the present invention relates to a liquid dispensing apparatus. The liquid dispensing apparatus may comprise:
a liquid storage defining a space, the liquid storage adapted storing a liquid, the liquid storage including a liquid dispensing opening and a movable wall for reducing the space,

1an intermediate storage space having a liquid inlet in fluid communication with the liquid storage for receiving liquid from the liquid storage, the intermediate storage variably defining volume and a specific maximum volume, the intermediate storage space further having a liquid outlet,
a nozzle having an inlet being in fluid communication with the liquid outlet of the intermediate storage space, the nozzle further having an outlet for dispensing the liquid to an intended surface
a specific amount of the liquid being transferred from the liquid storage to the intermediate storage space in a single operation.

In the liquid dispensing apparatus according to the present invention the liquid may further be dispensed from the intermediate storage space into the nozzle and from the nozzle to the intended surface in the single operation.

The liquid storage may at any given time define a volume. The movable wall allows the volume to change, in operation of the apparatus the volume is decreased as liquid is being dispensed from the apparatus to an intended surface, i.e. an eye of a patient.

The intermediate chamber is contemplated to allow delivery of a precise amount of liquid to the intended surface.

The nozzle atomises the liquid so that the liquid is not delivered to the intended surface as a jet or squirt but rather as a mist or cloud, as a jet most probably would make the person receiving the liquid in an eye uncomfortable. Mist pressure etc. is discussed in publications such as US 2003/0078551 and WO 03/002045. Reference is made to US 2003/0078551, which is hereby incorporated in the present specification by reference in its entirety for all purposes.

In the presently preferred embodiment of the present invention, liquid is dispensed from the liquid storage to the intended surface in a single operation, without requiring the person applying the liquid to perform any twist or turns of the device. This is contemplated to allow persons with e.g. arthritis or the like, to apply the liquid them selves. The apparatus is preferably operated by pushing or pulling operations only.

Further, the liquid dispensing apparatus may be made from a plastic material. Plastic material such as ABS, PP, PE or other may be used. Alternatively, the apparatus may be made from a metal material, such as stainless steel, aluminium or another material or composition of material. Still further, the apparatus may be made from a combination of materials, such as metal and plastic. The plastic material may be hard to allow the device to be handled and stored without causing damage to the liquid storage and thereby either polluting the liquid or spilling the liquid.

Advantageously, the liquid dispensing apparatus may be operated by pulling and/or pushing operations only, thereby allowing the liquid dispensing apparatus to be operated by a person having a disability or disease, such as arthritis and/or scleroses.

In one embodiment of the present invention, the nozzle may include 2 inlets. The number of inlets may be varied so as to allow the apparatus to dispense a specific amount of liquid in a specific period of time, thereby achieving a specific vapour pressure at the time of impact of the liquid at the intended surface, i.e. a human eye.

Specifically, the nozzle may be of a pressure-swirl type, but other types of nozzles may be used.

The nozzle may have a spray angle in the interval 10 degrees to 180 degrees, such as 40 degrees to 170 degrees, such as 50 degrees to 150 degrees, such as 60 degrees to 130 degrees, such as 70 degrees to 100 degrees, such as 10 degrees to 30 degrees, such as 30 degrees to 50 degrees, such as 50 degrees to 70 degrees, such as 70 degrees to 90 degrees, such as 90 degrees to 110 degrees, such as 110 degrees to 130 degrees, such as 130 degrees to 150 degrees, such as 150 degrees to 180 degrees, such as 100 degrees.

The spray angle may be selected by selection of nozzle type and/or geometric shape etc. The spray angle is also contemplated to have an influence on the pressure at which the liquid meets the intended surface.

Further specifically, flow of liquid from the liquid dispensing apparatus during operation may be less than or equal to 0.05 g/second. In other embodiment, the flow of liquid from the liquid dispensing apparatus during operation may be higher than 0.05 g/second. The speed or flow rate at which liquid is dispensed may be chosen to have a specific amount of liquid dispensed in a specific interval of time with a specific pressure at the outlet of the nozzle.

Still further, liquid dispensed from the liquid dispensing apparatus during operation may be approximately 0.05 ml. Higher or lower amounts of liquid may be dispensed in one operation. The amount of liquid dispensed may be selected or predefined by varying the size of the intermediate chamber, a larger intermediate chamber may allow for a greater amount of liquid to be dispensed, while a smaller intermediate chamber may allow a smaller or lesser amount of liquid to be dispensed.

The present invention advantageously further provides the apparatus with an interface part defining opposite openings for interfacing the liquid dispensing apparatus with an eye of a human. The interface part may define a longitudinal body with an inner space, such as the body having truncated cone geometry. In one embodiment of the present invention the interface part may define an increasing cross-section along a longitudinal axis defined by the interface part.

Still further the interface part may include a core material of a plastic material and a coating of a soft flexible material, such as silicone. Alternatively, a soft material may be applied, e.g. by 2-component die casting, or by use of other techniques.

Even still further the interface part may include a protruding part for engaging and substantially closing the tear duct of a human eye. Specifically, the protruding part may be a smooth and/or continuous enlargement of the interface part and/or the coating. Alternatively, the protruding part is a discontinuous enlargement of the interface part and/or the coating.

The interface part may define an opening substantially corresponding to the contour of a human eye.

In one embodiment, the liquid is a NaCl-water solution. In another embodiment, the liquid is water including a medical compound, such as a post-operation treatment compound and/or a disinfecting compound and/or antibiotic.

A user of a dispensing device according to the present invention advantageously does not need to direct a pointy object towards or even bring close to the eye. Bringing a pointy object close to the eye may cause the user to poke the eye and possibly damage the eye or at least cause discomfort for the user. Also, the user is only allowed to apply a complete dose.

A second aspect of the present invention relates to an apparatus for repetitive dispensing of a predetermined quantity of liquid. The apparatus comprises:
a cylindrical body having a first end and an opposite second end together defining an axis, the cylindrical body defining an inner space for storing the liquid, the first end of the cylindrical body having a restricted opening through which the liquid may pass in response to pressure applied thereto,
a plunger extending into the inner space from the second end,
a spring operatively connected to the plunger for moving the plunger toward the first end of the cylindrical body,
an interface body for interfacing with an human eye to which the predetermined quantity of liquid is applied, the interface body in fluid communication with the restricted opening, the interface body being formed by a soft, flexible material, the interface body including at least one projection extending along or parallel with the cylinder,
an circumferential encircling body defining a tubular geometry encircling the cylinder, the circumferential encircling body including at least one ramped cam, at least one of the ramped cams engaging a projection of the interface body, and
the ramped cam and the projection configured and positioned so that rotation and/or movement of the encircling body with respect to each other moves the plunger toward towards the first end in one step.

The restricted opening acts as or constitutes a nozzle that is contemplated to form the liquid being dispensed into a mist so that the liquid does not cause discomfort to the person using the apparatus according to the present invention.

The interface part in the presently preferred embodiment of the present invention defines an open-ended structure with one or more sidewalls, and preferably generally defines a cup-shaped structure. The cup preferably corresponds to the contour of a human eye and preferably engages the skin around the human eye in a way that no mist or liquid may be released outside or away from the eye.

The cylindrical body constitutes a container for storing a dispensing the liquid into or onto a human eye. In the presently preferred embodiment of the present invention, the cylindrical body may further include an intermediate chamber for receiving and storing a specific volume of the liquid received from the container, the intermediate chamber defining an inlet and an outlet, the inlet in fluid communication with the container, the outlet in fluid communication with the interface part.

The intermediate chamber may be used for pre-loading or storing an amount of liquid before it is released and dispensed.

The ramped cam and the projection engage each other when a pressure is applied to the apparatus when the user wishes to dispense an amount of liquid into an eye. The ramped cam and the projection is contemplated to ensure that an intended dose is released in one operation and the user is therefore not able to apply e.g. half a dose, or even more that one dose.

The plunger may be in contact with a mechanical restriction ensuring that the plunger is only moved one step at a time when released by the ramped cam and the projection.

In the intermediate chamber the specific volume may be 0.01 to 100 ml, such as 0.05 to 90 ml, such as 0.1 to 80 ml, such as 0.5 to 70 ml, such as 1 to 60 ml, such as 1.5 to 35 ml, such as 2 to 20 ml, such as 3 to 10 ml, such as 4 to 5 ml, such as 0.01 to 0.05 ml, such as 0.05 to 0.1 ml, such as 0.1 to 0.5 ml, such as 0.5 to 1 ml, such as 1 to 2 ml, such as 2 to 3 ml, such as 3 to 5 ml, such as 5 to 10 ml, such as 10 to 20 ml, such as 20 to 40 ml, such as 40 to 60 ml, such as 60 to 100 ml.

Depending on the size of the intermediate chamber and the size of the container, the apparatus may be used repeatedly, e.g. from 1 to several 100 times.

Advantageously, the interface part may include a coating made from a soft flexible material such as silicone and a core material of a plastic material. The core material may ensure that the interface part does not loose its shape after use.

More advantageously, the interface part or the coating may include a protruding part for engaging and substantially closing the tear duct of a human eye. As the interface part in it self may close off or temporarily seal of the eye, the tear duct may be engaged by a projecting or protruding part. Specifically, the protruding part may be a smooth and/or continuous enlargement of the interface part and/or the coating. Alternatively, the protruding part may be a discontinuous enlargement of the dispensing part and/or the coating.

The inner space defined in the container may be 0.1 to 100 ml, such as 2 to 75 ml, such as 5 to 50 ml, such as 10 to 35 ml, such as 20 to 25 ml, such as 0.1 to 5 ml, such as 5 to 15 ml, such as 15 to 25 ml, such as 25 to 35 ml, such as 35 to 45 ml, such as 45 to 65 ml, such as 65 to 85 ml, such as 85 to 100 ml.

In the presently preferred embodiment of the present invention a hard plastic material is preferably used to form one or more of the parts constituting the apparatus. Plastic materials used may be ABS, PP, PE, POM, Nylon, PC or combinations thereof. Not all parts need be made from the same material. The interface part, or cup, is preferably made from a silicone based material and/or coated with a soft material.

The features of the first aspect of the present invention may, of cause, be combined with any or all of the features of the second aspect of the present invention.

### The present invention is now to be further described with reference to the drawings in which

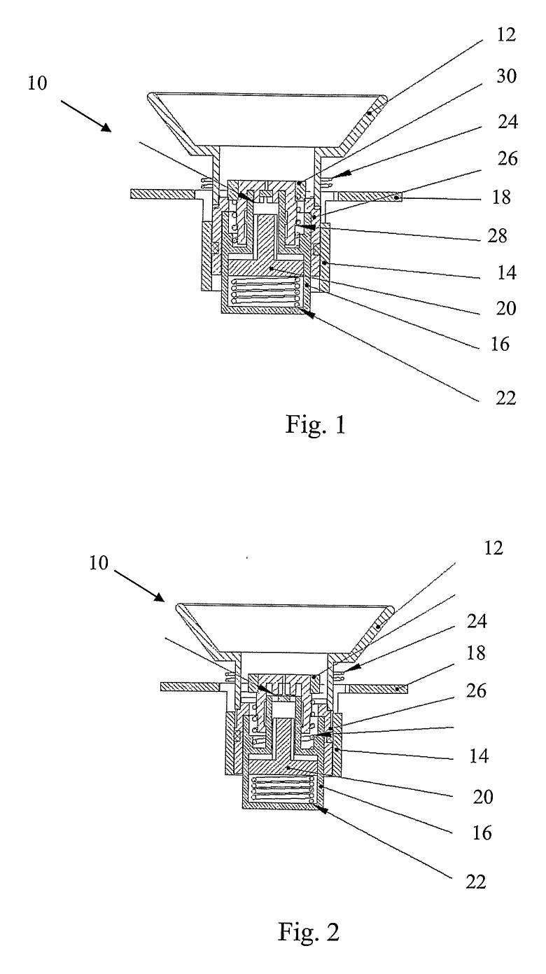

FIG. 1 is a schematic cut-through view of a device 10 according to the present invention. The device 10 comprises an interface part 12 connected to a body 14. The body 14 comprises a container for storing a liquid to be dispensed onto an intended surface, preferably a human eye. The body 14 includes a circumferential wall 18 extending perpendicularly from the body 14. The wall 18 is used by a person applying the liquid in the container 16 to the intended surface by pressing or holding the device using the wall 18, as will be discussed below.

Inside the container 16, a piston 20 is mounted. The piston is pressed by a spring 22 ensuring that the fluid inside the container 16 is stored at a pressure suitable for releasing the fluid from the container 16 and into or onto the intended surface. The piston 20 comprises a base part for interfacing with the spring 22 and a part extending into the container 16.

The spring 24 ensures that the interface part 12 is returned to the initial state after dispensing a specific amount of liquid from the container into a human eye. The spring 24 is in contact with the wall 18 and the bottom part of the interface part 12.

Inside the body 14, an encircling body 26 encircling the container 16 is located for ensuring that a specific amount of fluid is released from the container by not allowing the fluid to be dispensed continuously, but only in one irreversible operation, preferably being instant.

The operation of the encircling body 26 is discussed in relation to the following drawings.

An additional spring 28 pulls on the container 16 for bringing the container in closer contact with the interface part 12.

In FIG. 2, the interface part 12 is pressed towards the container 16, whereby a small volume or space is established between the container 16 and a block 30. The block 30 contains an aperture or opening constituting a nozzle for the release of the liquid. In the presently preferred embodiment of the present invention, the nozzle in the block 30 defines a cylindrical geometry, but may in alternative embodiments be constituted by other geometries, such as square, oblong, triangular or any other geometry. Also, more than one nozzle may be defined in the block 30.

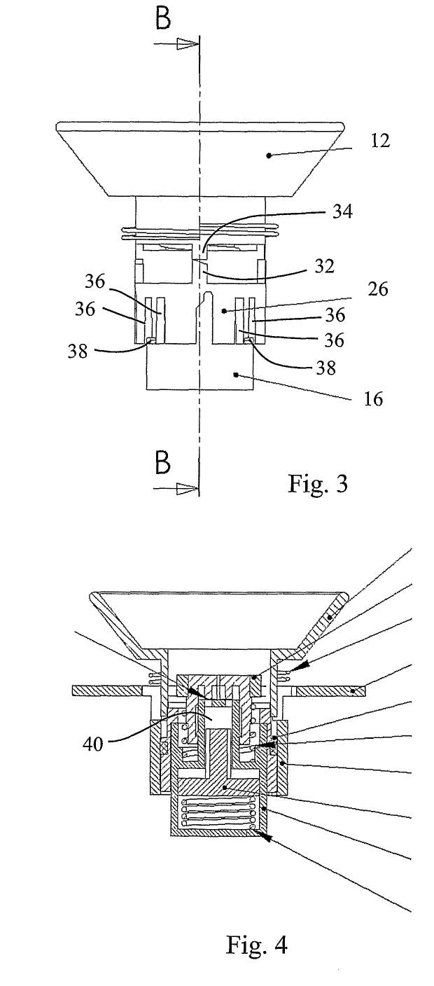

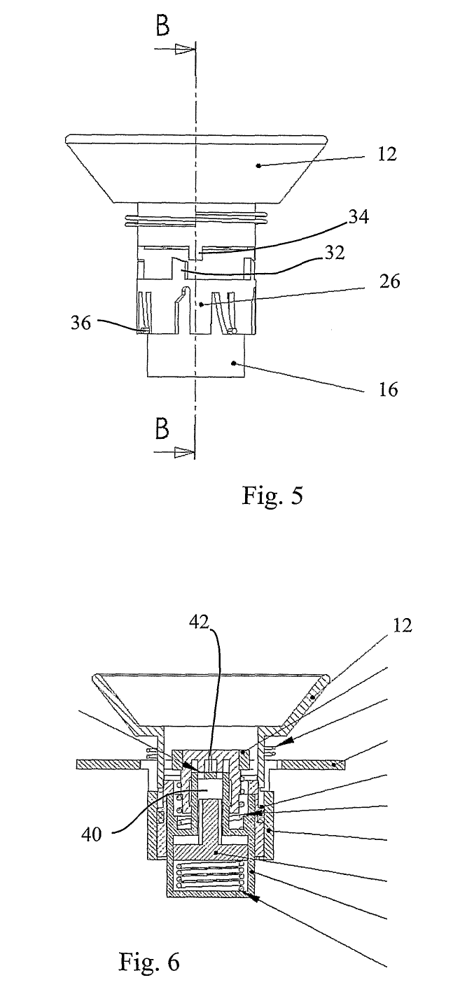

FIG. 3 is a schematic view of the interface part 12, the encircling body 26 and the container 16. As seen from FIG. 3, the encircling body 26 includes a protruding part 32 that, in the initial state, is in contact with a corresponding protruding part 34 of the interface 12. The protruding part 32 has a slanted edge so that when the interface part 12 is moved towards the encircling body 26, the encircling body 26 turns to the left and is able to proceed further towards the interface part 12. This is contemplated to enable a person using the device 10 to load or prepare the device 10 for a sudden release of the specific dosage or volume to the eye of a person without enabling the person to deliver e.g. half a dose, but only the full dose. The encircling body 26 further includes grooves or tracks 36 for co-operating with tracks or protruding parts 38 mounted to or being integral with the container 16. FIGS. 4 and 5 illustrate the device 10 in a compressed state before the release of the dose or volume. In this state, the user has depressed either the interface part 12 towards an eye or the circumferential wall 18 to a point where the protruding part 32 of the encircling body is almost not in contact with the protruding part 34 of the interface part 12 as illustrated in FIG. 5.

The state of the device 10 as illustrated in FIG. 4 also allows for the piston 20 to press the liquid stored in the container 16 into the space or volume 40 within the container 16.

The track 36 of the encircling body 26 ensures that the encircling body 26 is twisted or turned in relation to the interface part 12 and thereby allowing the protruding part 32 to glide off the protruding part 34 of the interface part 12.

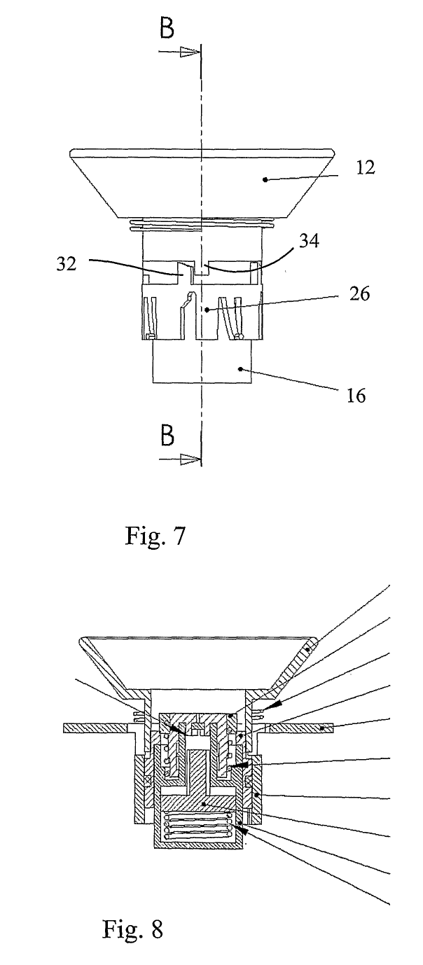

In FIG. 6, the device 10 is illustrated in a state, wherein the liquid stored in the volume 40 is dispensed or released through the nozzle 42. As the fluid or liquid is dispensed through the nozzle 42, the liquid is distributed or transformed into a mist travelling through the opening of the interface part 12 in a manner so that when the mist reaches the eye of the person using the device, the mist will not cause any discomfort to the person. The pressure of the liquid as it exits the nozzle 42 is preferably around 0 bar overpressure and one bar at the inlet of the nozzle. The state of the device as illustrated in FIG. 7 is after the release of the liquid from the chamber or volume 40. As seen from FIG. 7, the encircling body 26 is as close as possible to the interface part 12, as the encircling body 26 has been turned or twisted so that the protruding part 32 and 43 are no longer in contact.

FIG. 8 is a schematic illustration of the device 10, wherein the interface part 12 is pressed back to the initial state by the spring 24 so that the device is ready for use again.

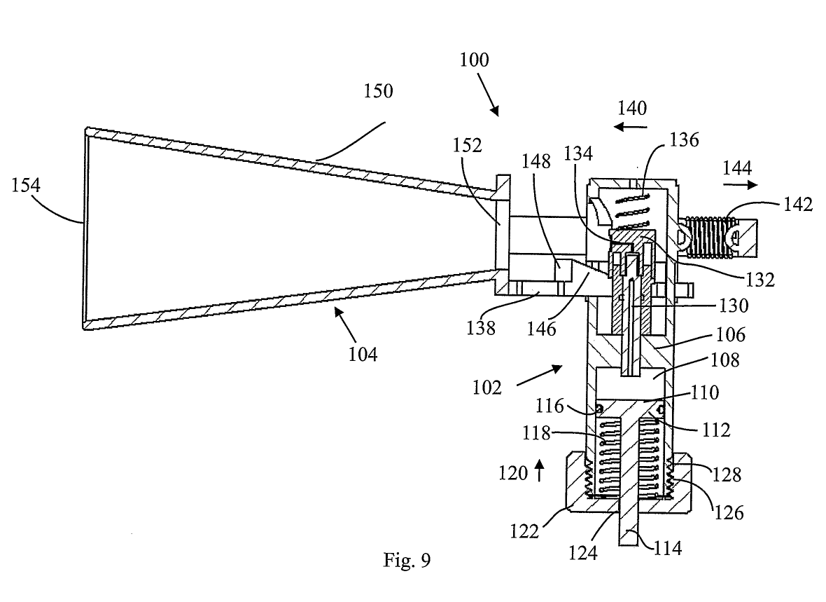

FIG. 9 is a schematic illustration of the second embodiment of the present invention designated the reference number 100. The device 100 comprised two parts, namely a dispensing part 102 and an interfacing part 104. The dispensing part 102 comprises a body 106 wherein a storage space 108 is defined. One of the walls in the storage space 108 is constituted by a piston 110 comprises a piston head 112 and a piston rod 114. The piston head 112 includes a sealing member 116. The sealing member 116 is in the presently preferred embodiment of the present invention constituted by a resilient ring made from a plastic material.

A liquid is stored in the storage space 108 and may be water, salt water or water with a medical compound solution.

A spring 118 excerpts a force on the piston head 112 in the direction of the arrow 120. The spring 118 rests against a cap 122 closing the open end of the body 106 at the storage space 108. The cap 122 includes an aperture 124 where through the piston rod 114 extends. The cap 122 and the body 106 may be formed so that the piston rod 114 does not extend beyond the exterior of the dispensing part 102.

The cap 122 includes a thread 126 engaging a thread 128 on the body 106.

A channel 130 connects the storage space 108 with a head 132 including a nozzle 134 where from liquid is dispensed. The head 132 is engaged by a spring 136 abutting a top part of the body 106.

The interfacing part 104 includes an arm 138 whereon the body 106 is slidedly mounted. The body 106 slides on the arm 138 in the direction of the arrow 140 during operation. FIG. 9 schematic illustrates the body in a start position. The spring 142 pulls the body 106 in the direction of the arrow 144 as will be discussed later.

The arm 138 includes a ramped cam 146 whereon the head 132 slides, whereas the body 106 slides in the direction of the arrow 140. The ramped cam 146 includes a flat part 148.

The interfacing part 104 is in the embodiment of the present invention illustrated in FIG. 9 a truncated cone 150 with two open ends 152 and 154. As the body 106 slides in the direction of the arrow 140 towards the open end 152, fluid is transferred from the storage space 108 to an intermediate space created or defined at the end of the channel 130 and the head 132. When the body 106 reaches its end position, atomised fluid is released through the nozzle 134 through the opening 152, whereby the atomised fluid travels through the interfacing part and intercepts the intended surface, i.e. the eye of a human. This operation will be illustrated further in the subsequent figures.

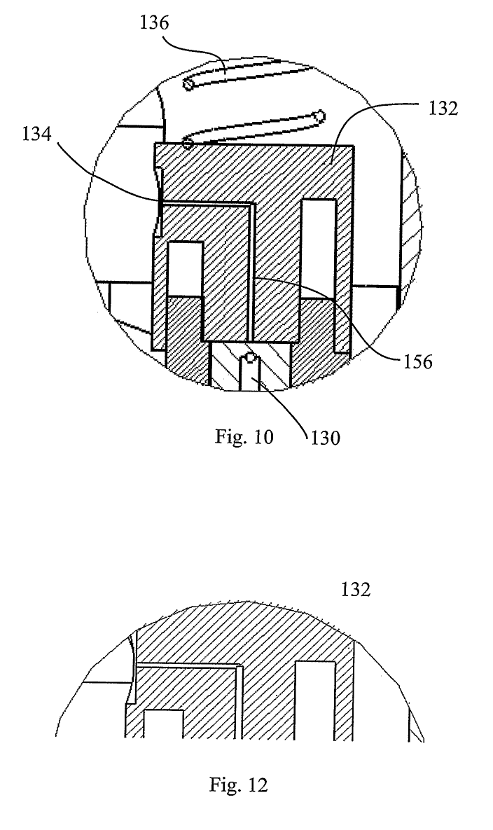

FIG. 10 is a zoomed view of a part of the device 100 illustrated in FIG. 9. FIG. 10 illustrates that head 132 abuts the end of the channel 130 as the device 100 is in its initial or starting state. The head 132 further includes a channel 136 where through fluid flows from the channel 130 to the intermediate storage chamber 158 and through the nozzle part 134.

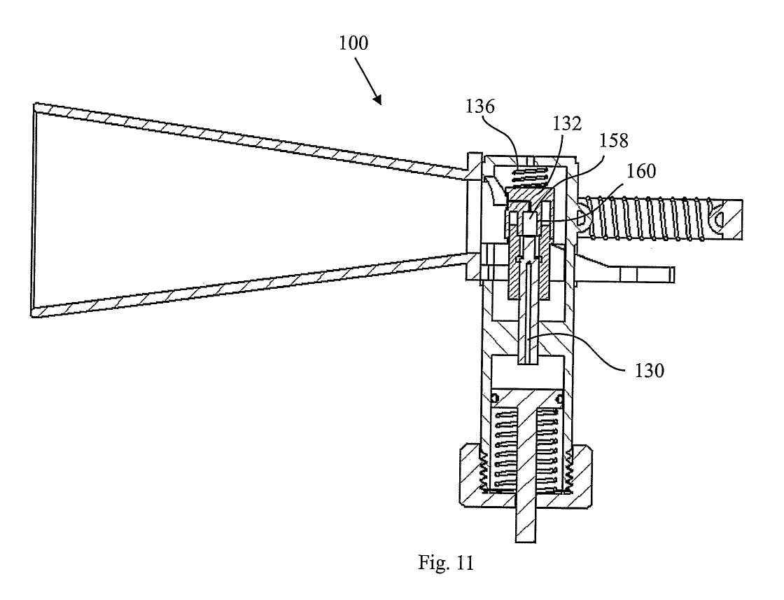

FIG. 11 is a schematic illustration of the device 100 in a state just prior to release of fluid from the intermediate storage space 158 defined at the end of the channel 130 and a cavity 160 defined in the head 132.

The spring 136 is in this position compressed relative to the position illustrated in FIG. 9.

FIG. 12 is a schematic zoomed view of the head 132 and illustrates the intermediate storage space 158 defined by the cavity 160 and the top of the body including the channel 130. As the head 132 is raised, a space is defined wherein the liquid is temporarily stored before it is transferred to the nozzle where from it is dispensed to the intended surface.

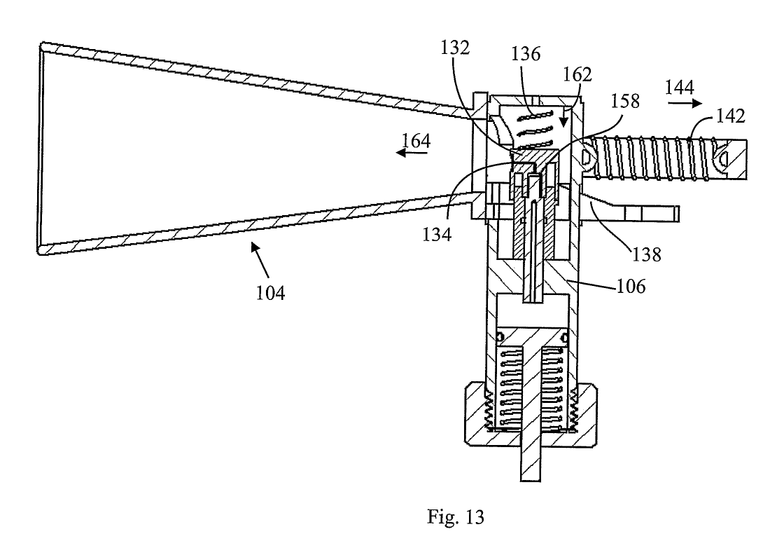

FIG. 13 is a schematic illustration of the device 100 after release of fluid from the intermediate chamber or storage space 158. The head 132 has passed the end of the flat part 148 of the ramped cam 146, and the spring 136 has pressed the head 132 in the direction of the arrow 162. The fluid has left the nozzle 134 in the direction of the arrow 164 towards the open end 154 of the interfacing part 104.

When the person using the device 100 releases his or her grip on the device 100, the spring 142 will pull the body 106 in the direction of the arrow 144. The arm 138 is in this embodiment flexible so that the end of the ramped cam 146 presses against the outer wall of the head 132 allowing the head 132 to remain in the position illustrated in FIG. 13 as the body travels to the initial or starting position illustrated in FIG. 9.

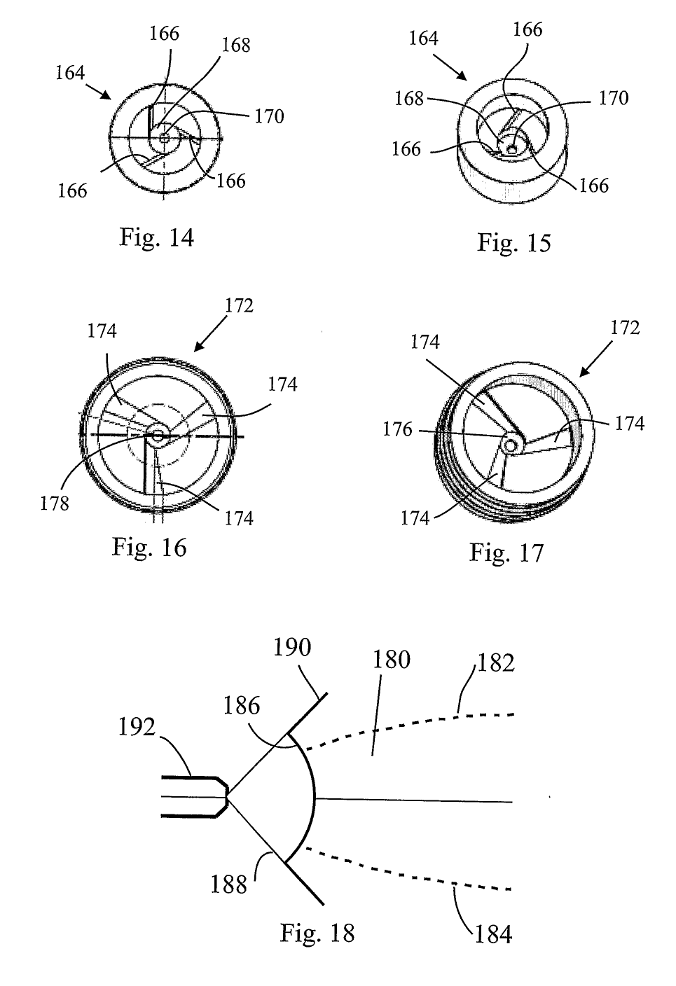

FIG. 14 is a schematic illustration of a nozzle 164 having three channels 166. Fluid flows from the outside of the nozzle 164 through the channels 166 into a swirl chamber 168 in the centre of the nozzle 164. An aperture 170 is formed in the nozzle 164 at the centre of the swirl chamber 168. The liquid flows through the channel 166 into the swirl chamber 168 and due to the geometrical configuration, the fluid starts to swirl inside the swirl chamber 168 and exits the swirl chamber 168 through the aperture 170.

FIG. 15 is a schematic perspective view of the nozzle 164 of FIG. 14.

FIG. 16 is a schematic top view of a second embodiment of a nozzle 172 having three channels 174. The channels 166 illustrated in FIG. 14 all had substantial equal cross section along the entire length of the channel 166. The channels 174 of the nozzle 172 have a decreasing cross section meaning that a liquid flowing through the channel 174 will have an increasing flow rate and/or pressure as the fluid flows through the channel 174 and into the swirl chamber 176 and out through the aperture 178.

FIG. 17 is a schematic perspective view of the nozzle 172 of FIG. 16 where it is seen that the swirl chamber 176 have a different geometry compared to the swirl chamber 168 of the nozzle 164 illustrated in FIG. 14 and 15. The swirl chambers 176 have a truncated cone shape meaning that the diameter of the swirl chamber decreases seen from the view of the fluid or liquid flowing from the channels 174 through the flow chamber 176.

The nozzles 164 and 172 are two examples of nozzles that may be used in connection with the device according to the present invention. The two nozzles are illustrated having three channels for receiving liquid or fluid to be dispensed through the openings of the nozzle. The width and/or cross section of the channels may be varied depending on the desired amount of fluid and the pressure and velocity of the atomised fluid exiting the nozzle as required. Also the number of channels may be varied so that the embodiments wherein a low amount of fluid is required only one channel could be used or channels having a small diameter thereby limiting the amount of fluid exiting in one period of time. The diameter of the apertures may also be varied as well as the shape of the swirl chamber in order to obtain a specific spray angle such as illustrated in FIG. 18 where a spray cloud 180, illustrated by the punctured lines 182 and 184. The spray angle 186 may be defined as the angle between the solid lines 188 and 190 measuring the spray angle 186 as the fluid or atomised fluid leaves the nozzle 192.

The figures schematically illustrates different embodiments of the present invention. Any feature or features of the embodiments may be combined. The illustrated embodiments are not to be considered limiting to the scope of the present invention but merely as examples on how to implement the teachings of the present invention.

The present invention may be characterised by the following points:

1. Apparatus for repetitive dispensing of a predetermined quantity of liquid comprising:
a cylindrical body having a first end and an opposite second end together defining an axis, said cylindrical body defining an inner space for storing said liquid, the first end of said cylindrical body having a restricted opening through which said liquid may pass in response to pressure applied thereto,
a plunger extending into said inner space from said second end,
a spring operatively connected to said plunger for moving said plunger toward said first end of said cylindrical body,
an interface body for interfacing with an human eye to which said predetermined quantity of liquid is applied, said interface body in fluid communication with said restricted opening, said interface body being formed by a soft, flexible material, said interface body including at least one projection extending along or parallel with said cylinder,
an circumferential encircling body defining a tubular geometry encircling said cylinder, said circumferential encircling body including at least one ramped cam, at least one of said ramped cams engaging a projection of said interface body, and
said ramped cam and said projection configured and positioned so that rotation and/or movement of said encircling body with respect to each other moves said plunger toward towards said first end in one step.

2. The apparatus according to point 1 wherein
said cylindrical body further includes an intermediate chamber for receiving and storing a specific volume of said liquid received from said container, said intermediate chamber defining an inlet and an outlet, said inlet in fluid communication with said container, said outlet in fluid communication with said interface part.

3. The apparatus according to point 1 or 2 wherein said specific volume is 0.01 to 100 ml, such as 0.05 to 90 ml, such as 0.1 to 80 ml, such as 0.5 to 70 ml, such as 1 to 60 ml, such as 1.5 to 35 ml, such as 2 to 20 ml, such as 3 to 10 ml, such as 4 to 5 ml, such as 0.01 to 0.05 ml, such as 0.05 to 0.1 ml, such as 0.1 to 0.5 ml, such as 0.5 to 1 ml, such as 1 to 2 ml, such as 2 to 3 ml, such as 3 to 5 ml, such as 5 to 10 ml, such as 10 to 20 ml, such as 20 to 40 ml, such as 40 to 60 ml, such as 60 to 100 ml.

4. The apparatus according to any of the points 1-3 wherein
said interface part is constituted by an open ended structure defining a cup substantially corresponding to the contour of a human eye.

5. The apparatus according to any of the points 1-4 wherein
said dispensing part includes a coating made from a soft flexible material such as silicone and a core material of a plastic material.

6. The apparatus according to any of the points 1-5 wherein
said interface part or said coating includes a protruding part for engaging and substantially closing the tear duct of a human eye.

7. The apparatus according to point 6 wherein
said protruding part is a smooth and/or continuous enlargement of said interface part and/or said coating.

8. The apparatus according to point 6 wherein
said protruding part is a discontinuous enlargement of said dispensing part and/or said coating.

9. The apparatus according to any of the points 1-8, wherein said inner space is 0.1 to 100 ml, such as 2 to 75 ml, such as 5 to 50 ml, such as 10 to 35 ml, such as 20 to 25 ml, such as 0.1 to 5 ml, such as 5 to 15 ml, such as 15 to 25 ml, such as 25 to 35 ml, such as 35 to 45 ml, such as 45 to 65 ml, such as 65 to 85 ml, such as 85 to 100 ml.

1. A liquid dispensing apparatus comprising:
a liquid storage space, said liquid storage space adapted for storing a liquid, said liquid storage space including a liquid dispensing opening and a movable wall for reducing said storage space;
an intermediate storage space having a liquid inlet in fluid communication with said liquid storage space for receiving liquid from said liquid storage space, said intermediate storage defining a variable volume, said intermediate storage space further having a liquid outlet;
a nozzle having an inlet being in fluid communication with said liquid outlet of said intermediate storage space, said nozzle further having an outlet for dispensing said liquid to an intended surface; wherein
a specific amount of said liquid is transferred from said liquid storage space to said intermediate storage space in a single operation.

2. The liquid dispensing apparatus according to claim 1, wherein said liquid is dispensed from said intermediate storage space into said nozzle and from said nozzle to said intended surface in said single operation.

3 The liquid dispensing apparatus according to claim 1, wherein said liquid dispensing apparatus is made from a plastic material.

4 The liquid dispensing apparatus according to claim 1, wherein said liquid dispensing apparatus is operable by pulling and/or pushing operations.

5. The liquid dispensing apparatus according to claim 1, wherein said nozzle includes two inlets.

6. The liquid dispensing apparatus according to claim 1, wherein said nozzle is of a pressure-swirl type.

7. The liquid dispensing apparatus according to claim 1, wherein said nozzle has a spray angle of 10 degrees to 180 degrees.

8. The liquid dispensing apparatus according to claim 1, wherein flow of liquid from said liquid dispensing apparatus during operation is no greater than 0.05 g/second.

9. The liquid dispensing apparatus according to claim 1, wherein the volume of the liquid dispensed from said liquid dispensing apparatus during operation is approximately 0.05 ml.

10. The liquid dispensing apparatus according to claim 1, wherein said apparatus further includes an interface part defining opposite openings for interfacing said liquid dispensing apparatus with an eye of a human.

11. The liquid dispensing apparatus according to claim 10, wherein said interface part defines a longitudinal body with an inner space.

12. The liquid dispensing apparatus according to claim 10, wherein said interface part defines an increasing cross-section along a longitudinal axis defined by said interface part.

13. The liquid dispensing apparatus according to any claim 10, wherein said interface part includes a core material of a plastic material and a coating of a soft flexible material.

14. The liquid dispensing apparatus according to claim 10, wherein said interface part includes a protruding part for engaging and substantially closing the tear duct of a human eye.

15. The liquid dispensing apparatus according to claim 14, wherein said protruding part is a smooth continuous enlargement of said interface part.

16. The liquid dispensing apparatus according to claim 14, wherein said protruding part is a discontinuous enlargement of said interface.

17. The liquid dispensing apparatus according to claim 10, wherein said interface part defines an opening substantially corresponding to the contour of a human eye.

18. The liquid dispensing apparatus according to claim 1, wherein said liquid is a NaCl-water solution.

19. The liquid dispensing apparatus according to claim 1, wherein said liquid is water including a medical compound selected from the group consisting of a post-operation treatment compound, a disinfecting compound, an antibiotic, and any combination thereof.
